(19) United States
(12) Patent Application Publication
(10) Pub. No.: US 2020/0163364 A1
(43) Pub. Date: May 28, 2020

(54) SHREDDED-MEAT-SHAPED PET FOOD AND PREPARATION METHOD THEREOF

(71) Applicant: Jiangsu Zhongheng Pet Articles Joint-Stock Co., Ltd., Yangcheng, Jiangsu (CN)

(72) Inventor: Bin QIU, Yancheng, Jiangsu (CN)

(73) Assignee: Jiangsu Zhongheng Pet Articles Joint-Stock Co., Ltd., Yangcheng, Jiangsu (CN)

(21) Appl. No.: 16/627,332

(22) PCT Filed: Jul. 7, 2017
(86) PCT No.: PCT/CN2017/092143

§ 371 Date: Dec. 29, 2019

(30) Foreign Application Priority Data
Jun. 29, 2017 (CN) 201710514236.0

(51) Int. Cl. A23K 40/25; A23K 10/20; A23K 10/30; A23K 20/147; A23K 20/158; A23K 20/174; A23K 20/163; A23K 40/20; A23K 10/18; A23K 10/12; A23K 20/26; A61K 31/702; A61K 35/644; A61K 36/185; A61K 36/82; A61K 33/34; A61K 33/30; A61K 31/015; A61K 31/737; A61K 31/593; A61K 35/56; A61K 35/60; A61K 36/55; A61K 31/07

(52) U.S. Cl. CPC A23K 40/25; A61K 31/07; A23K 10/30; A23K 20/147; A23K 20/158; A23K 20/174; A23K 20/163; A23K 40/20; A23K 10/18; A23K 10/12; A23K 20/26; A61K 31/702; A61K 35/644; A61K 36/185; A61K 36/82; A61K 33/34; A61K 33/30; A61K 31/015; A61K 31/737; A61K 31/593; A61K 35/56; A61K 35/60; A61K 36/55; A23K 10/20

(57) ABSTRACT

The present invention discloses a shredded-meat-shaped pet food which consists of the following raw materials in percentage by mass: 65 to 95% of plant-based protein, 0 to 20% of starch, 0 to 15% of animal raw materials, 1 to 7% of vegetables, 1 to 5% of fruits, 0.5 to 3% of beer yeast, 0.02 to 0.05% of potassium sorbate, 0.1 to 0.7% of salt, 0.3 to 0.5% of essences, 0.01 to 0.05% of phosphate and 0.02 to 0.03% of mixed vitamins. According to the technical solution of the present invention, the plant-based protein, meat, vegetables and fruits are prepared into the shredded-meat-shaped pet food by a mechanical puffing extrusion technology. The shredded-meat-shaped pet food in the technical solution of the present invention is a product having a fibrous shredded-meat shape, and thus has a very good sensory effect and palatability effect. As more and more people keep pets, such shredded-meat-shaped pet food has a broad development prospect and needs to be developed urgently to fill the market gap.

### TECHNICAL FIELD

The present invention relates to a pet food, and more particularly, to a shredded-meat-shaped pet food and a preparation method thereof, belonging to the field of pet food processing.

### BACKGROUND

The pet foods refer to foods for pets and small animals. They are high-grade animal foods between human foods and traditional livestock and poultry feeds, and mainly function to provide nutrient substances for the most basic life guarantee, growth and development, and health for various pets. The pet foods have the advantages of comprehensive nutrition, high digestion and absorption rates, scientific formula, standard qualities, convenience in feeding and use, capability to prevent certain diseases, and the like.

The standards for the pet foods cover moisture, proteins, crude fats, crude ash, crude fibers, nitrogen-free extracts, minerals, trace elements, amino acids, vitamins, etc., wherein the crude ash is a non-nutritive content, and crude fibers can stimulate gastrointestinal motility. Nutrition design and production of the pet foods must be guided by a pet nutritionist specializing in pet nutrition. Scientific and reasonable pet food standards should be formulated according to nutritional needs in comprehensive consideration of different growth stages of pets, their own physiques, and different seasons. When foods are purchased and used for pets, they should be selected according to the pet's own physiological characteristics and growth stages, and properly matched and fed.

The staple foods and snacks for pets in current pet foods are difficult to achieve a real meat-like fibrous effect. Most meats are difficult to preserve in a fibrous shredded-meat shape after being minced and chopped.

### SUMMARY

In view of the above defects, an object of the present invention is to provide shredded-meat-shaped foods, which provide novel pet foods for pets and pet owners.

Another object of the present invention is to provide preparation methods for the pet foods.

The objects of the present invention are realized by the following technical solutions:

a shredded-meat-shaped pet food consists of the following raw materials in percentages by mass (by weight):

| | |
|---|---|
| plant-based protein | 65-95%; |
| starch | 0-20%; |
| animal raw materials | 0-15%; |
| vegetables | 1-7%; |
| fruits | 1-5%; |
| Beer yeast | 0.5-3%; |
| potassium sorbate | 0.02-0.05%; |
| salt | 0.1-0.7%; |
| essences | 0.3-0.5%; |
| phosphate | 0.01-0.05%; and |
| mixed vitamins | 0.02-0.03%. |

More preferably, a shredded-meat-shaped pet food consists of the following raw materials in percentage by mass:

| | |
|---|---|
| plant-based protein | 70-90%; |
| starch | 1-15%; |
| animal raw materials | 1-10%; |
| vegetables | 1-7%; |
| fruits | 1-5%; |
| Beer yeast | 0.5-3%; |
| potassium sorbate | 0.02-0.05%; |
| salt | 0.1-0.7%; |
| essences | 0.3-0.5%; |
| phosphate | 0.01-0.05%; and |
| mixed vitamins | 0.02-0.03%. |

The plant-based protein is purified plant-based protein selected from at least one of soybean protein isolates, soybean protein concentrates, pea protein isolates, wheat protein extracts, and the like.

The vegetables are fresh vegetables selected from at least one of green vegetables, carrots, pumpkin, cucumbers, and the like.

The fruits are freeze-dried or heat-dried fruits selected from at least one of dried apples, pears, avocados, dried bananas, and the like.

The beer yeast (brewer yeast) is feed-grade beer yeast (brewer yeast).

The essences are selected from products obtained by decomposing and fermenting entrails and leftovers of animals, essences blended with aromatic substances and the like.

The starch is at least one of corn starch, potato starch, tapioca starch and other plant starch.

The animal raw materials are animal edible raw materials selected from at least one of livestock meat, livestock skin, livestock entrails and animal raw materials.

The shredded-meat-shaped pet food further comprises functional ingredients, accounting for 0.5-5% of the total; the functional raw material ingredients may include at least one category selected from a gastrointestinal nursing raw materials, an antioxidant free radical scavenging raw material, a joint nursing raw materials, and a fur nursing raw materials. Addition of the functional raw materials to the above formula convert them into functional products.

The gastrointestinal nursing raw materials are selected from at least one of fructo-oligosaccharides, isomalt oligosaccharides, hawthorn, honey, edible fibers, and the like.

The antioxidant free radical scavenging materials are selected from at least one of green tea powder, flavonoids, β-carotene, and other antioxidation raw materials.

The joint nursing raw materials are selected from at least one of chondroitin, glucosamine sulfate/hydrochloride, bone meal, vitamin D3, and other raw materials having a joint protection function.

The fur nourishing raw materials are selected from at least one of foods containing unsaturated fatty acids such as salmon meat and flaxseed meal/oil, and vitamin A, vitamin B-based raw materials having a hair nourishing function.

The meat-shaped pet food has a shape selected from at least one of a bone shape, a sheet/plate shape, a rod shape, a filament/string shape, a granular shape, and a polygonal shape.

Embodiments of the present invention may be made commercially available on the market.

A preparation method for the shredded-meat-shaped pet food comprises the following steps:

(1) naturally thawing frozen animal raw materials at 0-4° C., and then grinding the animal raw materials into a paste with a meat grinder;

(2) weighing the various raw materials according to production proportions; then placing all the raw materials in a stirring machine, and stirring for 1 to 5 minutes, and adding water under stirring (water is 25-50% of the total amount of raw materials);

(3) placing the stirred raw materials in a plastic bag for 1 hour, such that water is sufficiently and uniformly absorbed by the raw materials;

(4) starting a dedicated bulking machine: setting the temperature of a first zone to 75-85° C., the temperature of a second zone to 125-145° C., and the temperature of a third zone to 150-160° C.;

(5) starting the bulking machine after the temperatures are reached; adjusting a screw speed of a main machine and a rotational speed of a feeder, such that the raw materials are subjected to physical and chemical reactions at high temperatures and pressure to form a shredded-meat-shaped tissue structure, thereby obtaining a product that achieves a predetermined effect;

(6) treating the product with molds of various shapes, cooling, and cutting into shapes; naturally air dried over night; and

(7) re-watering the product by controlling the moisture of the product at a ratio of 25-35%, to form a shredded-meat-shaped pet food; and seasoning, packaging and sterilizing to obtain a finished product.

After monitoring, the nutritional contents of this product are: protein >50%; fat >1%; crude fiber <4%; ash <5%; and moisture <35%. The surface of the product has a shredded-meat shape. The product has an apparent shredded-meat (meat fiber) shape after being broken apart.

According to the technical solution of the present invention, a shredded-meat-shaped pet food is prepared from plant-based protein, meats, vegetables and fruits through a mechanical puffing extrusion process. By controlling the water contents, the temperatures of the bulking machine and the pressure of the feeder, a fibrous shredded-meat product is prepared. Such products have very good sensory effects and palatability effects. As more and more people keep pets, the shredded-meat-shaped pet foods have a broad development prospect and needs to be developed urgently to fill the market gap.

Compared with the existing pet foods, the products prepared by the technical solution of the present invention have obvious shredded-meat sensory effects (meat fiber feels). In the product formula, nutrition is relatively comprehensive. The products are rich in vitamins, minerals, and amino acids, with a protein content of up to 70%, which can meet the protein nutritional needs of pets. The plant-based protein in the products are more easily digested and absorbed after high temperature curing. The plant-based protein contain less fats and are rich in cellulose and other beneficial ingredients helpful for protecting pets' cardiovascular vessels and lowering cholesterol. The prices of the plant-based protein in the main raw materials are much lower than the prices of ordinary pet foods such as preserved meat, about ½ thee price of ordinary dried chicken meat and ⅓ of beef preserved meat, and therefore, the plant-based protein have obvious price advantages.

### DETAILED DESCRIPTION

The technical features of the present invention will be further described below with reference to specific examples.

The following preparation method is adopted in Examples 1-6 of the present invention:

(1) naturally thawing the frozen animal raw materials at 0-4 degrees for 12 hours, and then grinding the animal raw materials into ground meat or meat paste with a meat grinder;

(2) weighing various raw materials according to production proportions, and processing plant-based protein; then placing all the raw materials in a stirring machine, and stirring for 1 to 5 minutes; and adding water under stirring according to 25-50% of the total amount of raw materials;

(3) placing the stirred raw materials in a plastic bag for 1 hour, such that water is sufficiently and uniformly absorbed by the raw materials;

(4) starting a dedicated bulking machine: setting the temperature of a first zone to 75-85° C., the temperature of a second zone to 125-145° C., and the temperature of a third zone to 150-160° C.;

(5) starting the bulking machine after the temperatures are reached; adjusting a screw speed of a main machine and a rotational speed of a feeder, such that the raw materials are subjected to physical and chemical reactions at high temperature and pressure to form a shredded-meat-shaped tissue structure, thereby obtaining a product that achieves a predetermined effects;

(6) treating the product with molds of various shapes, cooling, and shearing for shaping; naturally airing over night; and

(7) re-hydrating the product by controlling the moisture of the product at a ratio of 30%, to form a shredded-meat-shaped pet food; and seasoning, packaging and sterilizing to obtain a finished product.

Proportions in Examples 1-6:

| | Example 1 | Example 2 | Example 3 | Example 4 | Example 5 | Example 6 |
|---|---|---|---|---|---|---|
| Plant-based protein | 65 | 70 | 75 | 80 | 85 | 87 |
| Starch | 20 | 7 | 12 | 7 | 9 | 5 |
| Animal raw materials | 10 | 10 | 5 | 5 | 2 | 2 |
| Vegetables | 1 | 7 | 3 | 3 | 2 | 1 |
| Fruits | 1 | 3 | 3 | 3 | 1 | 1 |
| Beer yeast | 2 | 2 | 1 | 1 | 0.5 | 3 |
| Potassium sorbate | 0.02 | 0.05 | 0.05 | 0.05 | 0.05 | 0.02 |
| Salt | 0.6 | 0.5 | 0.5 | 0.4 | 0.1 | 0.6 |
| Essences | 0.3 | 0.4 | 0.4 | 0.5 | 0.3 | 0.3 |
| Phosphate | 0.05 | 0.03 | 0.03 | 0.02 | 0.02 | 0.05 |
| Mixed vitamins | 0.03 | 0.02 | 0.02 | 0.03 | 0.03 | 0.03 |

Proportions in Examples 7-9 and Comparative Examples 1-3:

| | | | | Comparative | Comparative | Comparative |
|---|---|---|---|---|---|---|
| | Example 7 | Example 8 | Example 9 | example 1 | example 2 | example 3 |
| Plant-based protein | 88 | 90 | 95 | 60 | 50 | 96 |
| Starch | 3 | 3 | 0 | 15 | 20 | 1 |
| Animal raw materials | 3 | 1 | 1 | 12 | 15 | 0.5 |
| Vegetables | 2 | 1 | 1 | 7 | 8 | 0.5 |
| Fruits | 2 | 3 | 1 | 3 | 3 | 0.5 |
| Beer yeast | 1 | 1 | 1 | 2 | 3 | 0.5 |
| Potassium sorbate | 0.05 | 0.05 | 0.05 | 0.05 | 0.05 | 0.05 |
| Salt | 0.5 | 0.5 | 0.4 | 0.5 | 0.5 | 0.4 |
| Essences | 0.4 | 0.4 | 0.5 | 0.4 | 0.4 | 0.5 |
| Phosphate | 0.03 | 0.03 | 0.02 | 0.03 | 0.03 | 0.02 |
| Mixed vitamins | 0.02 | 0.02 | 0.03 | 0.02 | 0.02 | 0.03 |

The products of Examples 1-9 are assayed: the nutritional components thereof are as follows: protein >50%; fat >1%; crude fiber <4%; ash content <5%; water content <35%. The surfaces of the products have shredded-meat shapes. The products also have apparent shredded-meat shapes after being broken apart. From the appearance, the products look no different from real animal meat products, and have good sensory effects and palatability effects. While Comparative Examples 1-3 can meet the standards of nutritional ingredients such as protein and fat, but are not shredded-meat-shaped in appearance.

In the preparation methods in Examples 1-9: if the temperature range in step (4) is lower than 75° C. or greater than 160° C.; in step (7), the re-watering ratio is greater than 35% or less than 25%; although there are no difference in the nutritional components such as proteins and fats, no shredded-meat shape in appearance is formed.

1. A shredded-meat-shaped pet food, consisting of the following raw materials in percentage by mass:

| | |
|---|---|
| plant-based protein | 65-95%; |
| starch | 0-20%; |
| animal raw materials | 0-15%; |
| vegetables | 1-7%; |
| fruits | 1-5%; |
| Beer yeast | 0.5-3%; |
| potassium sorbate | 0.02-0.05%; |
| salt | 0.1-0.7%; |
| essences | 0.3-0.5%; |
| phosphate | 0.01-0.05%; and |
| mixed vitamins | 0.020.03%. |

2. The shredded-meat-shaped pet food according to claim 1, consisting of the following raw materials in percentage by mass:

| | |
|---|---|
| plant-based protein | 70-90%; |
| starch | 1-15%; |
| animal raw materials | 1-10%; |
| vegetables | 1-7%; |
| fruits | 1-5%; |
| Beer yeast | 0.5-3%; |
| potassium sorbate | 0.02-0.05%; |
| salt | 0.1-0.7%; |
| essence | 0.3-0.5%; |
| phosphate | 0.01-0.05%; and |
| mixed vitamins | 0.02-0.03%. |

3. The shredded-meat-shaped pet food according to claim 1, wherein the plant-based protein is a purified plant-based protein selected from at least one of soybean protein isolate, soybean protein concentrate, pea protein isolate, and wheat protein extract.

4. The shredded-meat-shaped pet food according to claim 1, wherein the vegetables are fresh vegetables selected from at least one of green vegetables, carrots, pumpkin, and cucumber.

5. The shredded-meat-shaped pet food according to claim 1, wherein the fruits are freeze-dried or heat-dried fruits selected from at least one of dried apples, pear, avocado, and dried bananas.

6. The shredded-meat-shaped pet food according to claim 1, wherein the essences are selected from at least one of products obtained by decomposing and fermenting entrails and leftovers of animals, and essences blended with aromatic substances.

7. The shredded-meat-shaped pet food according to claim 1, wherein the starch is at least one of corn starch, potato starch, and tapioca starch.

8. The shredded-meat-shaped pet food according to claim 1, wherein the animal raw materials are animal edible raw materials selected from at least one of livestock meat, livestock skin, and livestock entrails.

9. The shredded-meat-shaped pet food according to claim 1, further comprising functional ingredients, accounting for 0.5-5 wt % of a total; the functional raw material ingredients include at least one of gastrointestinal nursing raw materials, antioxidant free radical scavenging raw materials, joint nursing raw materials, and fur nourishing raw materials.

10. The shredded-meat-shaped pet food according to claim 9, wherein the gastrointestinal nursing raw materials are selected from at least one of fructo-oligosaccharides, isomalt oligosaccharides, hawthorn, honey, and edible fiber.

11. The shredded-meat-shaped pet food according to claim 9, wherein the antioxidant free radical scavenging raw materials are selected from at least one of green tea powder, brass, and β-carotene.

12. The shredded-meat-shaped pet food according to claim 9, wherein the joint nursing raw materials are selected from at least one of chondroitin, glucosamine sulfate/hydrochloride, bone meal, and vitamin D3.

13. The shredded-meat-shaped pet food according to claim 9, wherein the fur nourishing raw materials are selected from at least one of foods containing unsaturated fatty acids such as salmon meat, flaxseed meal/oil, and vitamin A, vitamin B-based raw materials having a hair nourishing function.

14. The shredded-meat-shaped pet food according to claim 1, wherein the meat-shaped pet food has a shape selected from at least one of a bone shape, a sheet shape, a rod shape, a filament-shape, a granular shape, and a polygonal shape.

15. A preparation method of the shredded-meat-shaped pet food according to claim 1, comprising the following steps:
(1) naturally thawing frozen animal raw materials at 0-4° C., and then grinding the animal raw materials into meat paste with a meat grinder;
(2) weighing various raw materials according to production proportions; then placing all the raw materials in a stirring machine, and stirring for 1 to 5 minutes; and adding water under stirring, wherein water added is 25-50% of a total amount of raw materials;
(3) placing the stirred raw materials in a plastic bag for 1 to 3 hours, such that water is sufficiently and uniformly absorbed by the raw materials;
(4) setting a bulking machine: setting a temperature of a first zone to 75-85° C., a temperature of a second zone to 125-145° C., and a temperature of a third zone to 150-160° C.;
(5) starting the bulking machine after the temperatures are reached; adjusting a rotational speed of a feeder and a rotational speed of a main machine to form a pressure of 4-7 MPa at a die head of the bulking machine, such that the raw materials are subjected to physical and chemical reactions at high temperature and pressure to form a shredded-meat-shaped tissue structure, thereby obtaining a product that achieves a predetermined effect;
(6) treating the product with molds of various shapes, cooling, and cutting out the various shapes; naturally air drying; and
(7) re-hydrating the product by controlling a moisture of the product at a ratio of 25-35%, to form a shredded-meat-shaped pet food; and seasoning, packaging, and sterilizing to obtain a finished product.
